(19) United States
(12) Patent Application Publication
(10) Pub. No.: US 2007/0107732 A1
(43) Pub. Date: May 17, 2007

(54) Oro-nasal mask

(76) Inventors: Michael Robert Dennis, Salisbury (GB); Christopher John Hindmarsh, Salisbury (GB); Laurence Anthony Pears, Salisbury (GB); James Nightingale, Halesowen (GB)

Correspondence Address: JOHN S. PRATT, ESQ;KILPATRICK STOCKTON, LLP, 1100 PEACHTREE STREET, ATLANTA, GA, 30309, US

(21) Appl. No.: 10/569,590

(22) PCT Filed: Sep. 1, 2004
(86) PCT No.: PCT/GB04/03744

§ 371 Date: Sep. 25, 2006

(30) Foreign Application Priority Data
Sep. 1, 2003 (GB) 0320319.7

(51) Int. Cl. A62B 18/02; A61M 15/08

(52) U.S. Cl. 128/206.21; 128/207.18

(57) ABSTRACT

There is provided an oro-nasal mask for use in a respirator, comprising a resilient material which mask is capable of recovering its original shape on release from a collapsed. A compact, emergency respirator including the mask is also provided.

The present invention is generally concerned with a collapsible oro-nasal mask and respirator suitable to prevent the inhalation of harmful chemicals, biological materials, particulates and/or radionuclides. The invention is particularly, but not exclusively, directed to a compact emergency respirator which can be conveniently carried or stored on, or in the vicinity of, the person.

Respirators are used throughout the world to protect against noxious and/or toxic agents such as chemical and biological warfare agents and industrial chemicals. In the current climate of concern and alert regarding the release of such agents or chemicals, there is a need for a quasi-disposable, emergency respirator of compact size which allows safe exit from a contaminated area and can be carried, for example, in a clothes pocket or on a belt, or stored in a desk drawer.

Although quasi-disposable emergency respirators are known, their compactness is often limited in that the oro-nasal mask has a certain degree of rigidity which prevents optimum carriage or storage. The present invention generally aims to improve upon this situation by providing for a more compact emergency respirator.

Accordingly, in a first aspect, the present invention provides an oro-nasal mask for use in a respirator, which mask comprises a resilient material and is capable of recovering its original shape on release from a collapsed form.

The mask may define air inlet and outlet means. In one embodiment, the mask simply defines apertures which can be connected to or overlaid with a suitable filter material or canister.

The air inlet means may, however, comprise air guidance means suitable for connecting to a filter canister. The air inlet guidance means preferably also comprises the resilient material of the rest of the mask.

The air outlet means may comprise air guidance means suitable for guiding exhaled air from a respirator to the atmosphere. It may, in particular, define a dead space for the respirator. The air outlet guidance means preferably also comprises the resilient material of the rest of the mask.

Advantageously, the resilient material capable of recovering shape from a collapsed form may be resistant to chemical agents. A preferred material comprises silicone rubber. The mask, optionally including air inlet and outlet guidance means, may be manufactured as a single piece by any suitable technique, for example, by injection moulding and conveniently folded for storage.

Oro-nasal masks of the prior art rely on a force component aligned with a central front axis for an effective seal. This force component is enabled by the rigidity of the mask, and in particular, the dead space of the air outlet guidance means. The force component typically arises by the action of a head strap and results in the peripheral sealing edge of the mask being pushed evenly against the face.

Although the air outlet guidance means of the mask may comprise a rigid material, in the preferred embodiment it is collapsible and defines a dead space which also recovers its shape on release from a collapsed form.

In this embodiment, the mask cannot rely on the transmission of forces mentioned above for an effective seal. The mask may, however, provide for an effective seal through one or more rigid support elements provided at or adjacent its peripheral edge. Preferably, the one or more supporting elements are provided on opposing lateral sides of the mask.

A suitable support element may, for example, comprise a flat, elongate plate or solid object shaped to conform to the contours of the side of the mask to which it is attached. Advantageously, the support element may comprise a filter canister.

The support element or elements provide a sealing force acting directly at or adjacent the sealing edge of the mask. The force may be applied by the action of a head strap or straps attached to the support elements.

It will be understood that, particularly where support elements are provided on opposing lateral sides of the mask, a substantial component of force acts perpendicularly to the adjacent surface of the face whereby to push the adjacent peripheral sealing edge of the mask against the face. A component of force also acts parallel to the face and provides for the remainder of the periphery and sealing edge of the mask, normally over the bridge of the nose and across the chin, to be pulled taut whereby to effectively seal these areas.

The flexibility of these parts of the mask enable it to conform to different face shape and sizes and may reduce the number of mask sizes necessary to service a population compared with prior art oro-nasal masks.

In a second aspect, the present invention provides a respirator including an oro-nasal mask according to the first aspect of the invention. Preferably, the respirator includes an oro-nasal mask incorporating the collapsible air inlet and outlet guidance means as mentioned above. However, it will be understood that the respirator or oro-nasal may provided with rigid air inlet and outlet guidance means.

In a preferred embodiment, the support elements comprise filter canisters provided on opposing lateral sides of the mask. The filter canisters are preferably slim-line, rectangular canisters having depth not exceeding 2 cm, or most preferably, 1 cm.

Suitable filter canisters may provide a ridge or wall on an outer surface for contacting the peripheral edge of the filter canister. Preferably, however, the support means includes means permitting folding of the filter canisters. Most preferably, the support means permit folding of the canisters whereby the major axis of each filter canister lies substantially within the same place.

It will be understood that the filter means may be folded between a first position in which the respirator is relatively compact and a second position in which it is not.

Suitable support means permitting folding of filter canisters, for example, comprise a hinged bracket optionally including a locking mechanism. The locking mechanism may lock the canisters such when the respirator is ready for use. The locking mechanism may provide permanent locking whereby to discourage second use of the respirator. Alternatively, it may comprise releasable locking means, such as a latch means.

Folding of the filter canisters may be assisted by a double-hinged cross-piece spanning the gap between filter canisters. The cross-piece may be provided with an abutment means controlling the extent of folding in desired and/or undesired directions.

Advantageously, the cross-piece defines means, for example an aperture, securing the air outlet guidance means. However, the air outlet guidance means may be secured elsewhere within the respirator.

As is usual in the art, the air outlet guidance means may include a one-way valve and define a dead space which may be covered, for example, by a gauze, filter or other paper, electrostatic filter materials or mechanical devices that reduce, and possibly filter, any air flow from the atmosphere into the dead space. A one-way valve may also be provided at the air inlet guidance means.

Preferably, the respirators of the present invention include a collapsible hood comprising a transparent material and, optionally, a neck seal. Most preferably the hood comprises a flexible material or materials which protect against chemical and/or biological warfare agents. A suitable hood comprises one or other of the flexible and transparent laminate materials mentioned in our international patent application WO 03/037443 A1.

It will be apparent that the oro-nasal mask of the present invention permits a respirator having various elements which can achieve a compact configuration for carriage and/or storage but in use provides effective protection against exposure to chemical and biological warfare agents. The compact configuration is especially facilitated by the provision of a collapsible dead space and support elements for sealing the mask utilising slim-line filter canisters and a folding mechanism. The compact configuration may, in particular, be achieved and retained by packaging until such time as the respirator is needed.

Accordingly, in a further aspect, the present invention provides a package including the respirator or mask of the first and second aspects, in which the respirator and/or mask is held in compact form.

The respirator may comprise any combination of the above mentioned features—with or without support elements permitting folding of the filter means.

Preferably, however, the respirator comprises a collapsible oro-nasal mask including support means provided on opposing lateral sides of the mask in which each support means comprise a filter canister and means permitting folding of the filter canisters. In this embodiment, the respirator may be substantially flat-packed such that the major axis of each filter canister lies substantially within the same plane.

Suitable packaging comprises vacuum pack bags or shrink wrap. Advantageously, the respirator may be packaged under substantially clean and/or sterile conditions. Optionally, the packaging may include instructions for use of the respirator.

It will be appreciated that the flat-packed respirator according to the present invention has a compact size, limited only by the dimensions of the filter canister, which permits easy carriage and/or storage of the respirator.

The present invention will now be described, by way of example, with reference to the accompanying drawings in which

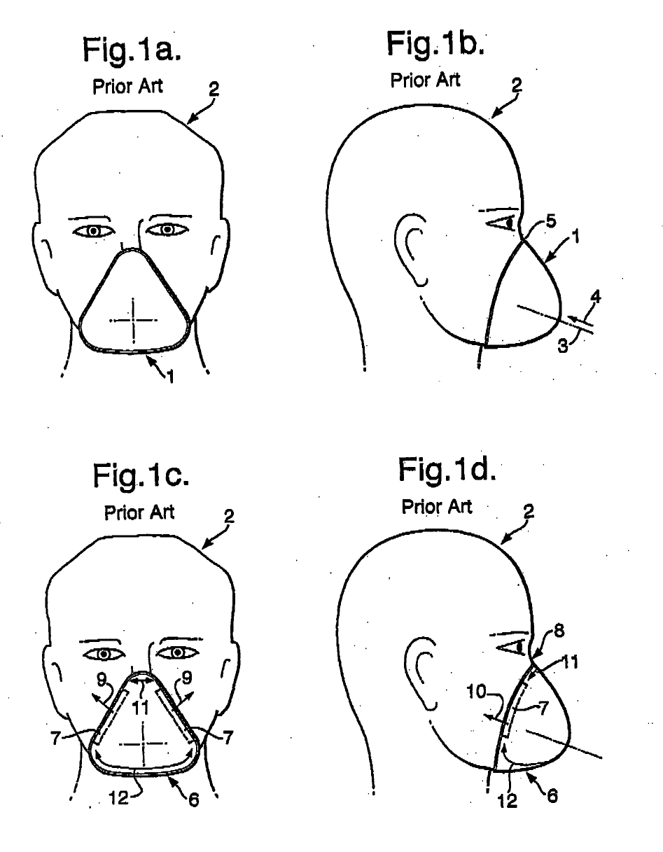

FIGS. 1 a) to d) schematically illustrate sealing forces of prior art oro-nasal masks and sealing forces of a preferred ora-nasal mask according to the present invention;

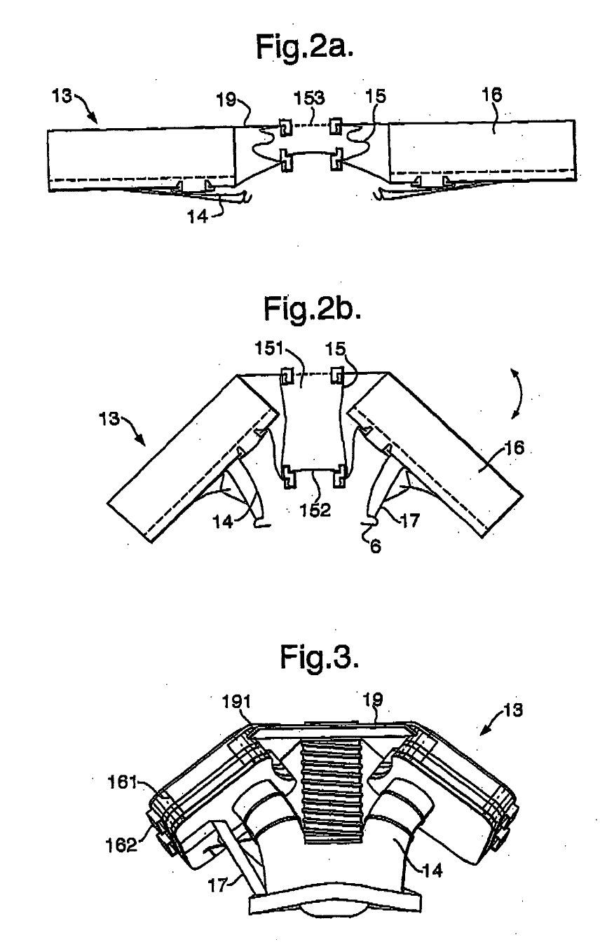

FIGS. 2 a) and b) are schematic illustrations of a respirator according to the present invention in ready to use form;

FIG. 3 is a perspective view of part a preferred respirator according to the present invention in ready to use form;

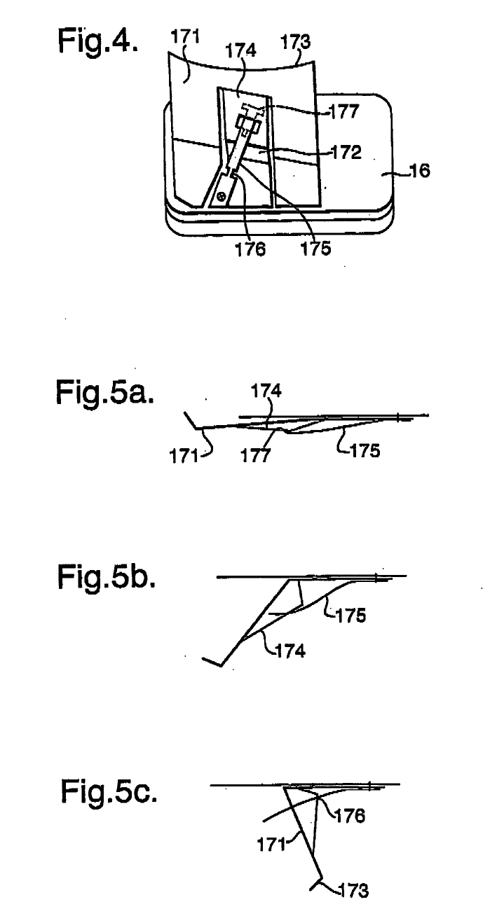

FIG. 4 is a perspective view of a preferred support element according to the present invention;

FIGS. 5 a) to c) schematically illustrate the folding mechanism of the support element of FIG. 4; and

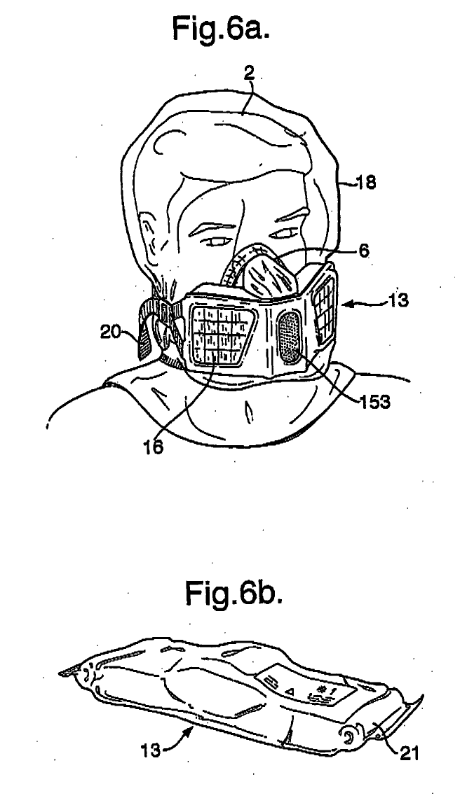

FIGS. 6 a) and b) are perspective views showing the preferred respirator in packaged in use and in packaged compact form.

Referring now to FIGS. 1 a) and b) the rigidity of a prior art oro-nasal mask 1 leads to a component of force 4 provided by a head strap (not shown) which acts in alignment with a central front axis 3 to the mask to secure its peripheral and sealing edge 5 to the face of the wearer 2.

Referring now to FIGS. 1 c) and d), the collapsible oro-nasal mask 6 of the present invention includes rigid support elements 7 disposed adjacent the edge 8 of opposing lateral sides of the mask. A component force (arrow 9) provided by a head strap (not shown) acts on the mask in a plane substantially perpendicular to the adjacent part of the face so that the sealing edge 8 immediately adjacent support elements 7 is pushed against the face. A component force (arrow 10) also acts on the mask substantially parallel to the adjacent part of the face so as to pull the remainder of the sealing edge 8 taut across the face (arrows 11 and 12).

Having regard now to FIGS. 2 b) and 3, a respirator, generally designated 13, comprises a collapsible silicone rubber oro-nasal mask 6 including integrally formed collapsible air inlet tubes 14 and an integrally formed collapsible air outlet tube 15.

The respirator utilises slim-line canisters 16 and split hinged brackets 17 as supporting elements for the oro-nasal mask. Filter canister 16 is connected to the air inlet tube 14 and includes a detachable lid portion 161 carrying lugs 162 provided for the attachment of a head strap (not shown). A hood 18 (see FIG. 8) is engaged by lid portion 161 of each filter canister 16.

Air outlet tube 15 defines a dead space 151 above a one-way outlet valve 152 and is secured to an aperture defined in a cross-piece 19. The cross-piece 19 comprises a double hinged bracket having limbs 191 secured to lid portion 161 of each filter canister 16. A wedge piece (not shown) provided on the inward facing surface of the cross-piece which includes the aperture stays limbs 191 so as to limit the extent of inward folding of the filter canisters. A gauze or similar filter material 153 secured to the cross-piece covers the outlet of tube 15.

Referring now to FIG. 4, split hinged bracket 17, shown partially collapsed, comprises a first single hinged portion 171 and a second double hinged portion 172. A first limb of single hinged portion defines a surface 173 adhering to the peripheral sealing edge 8 of mask 6. A second limb is secured to the underside of the filter canister 16.

Double hinged portion 172 is integral to the first limb of single hinged portion and provides a first limb 174 defining an aperture having a recess. A second limb is adhered to the filter canister 16. The aperture receives a strut element 175 having a lower portion secure to second limb of the double hinged portion, a constricted portion 176 and a tab portion 177.

Referring now to FIGS. 2 b) and FIGS. 5 a) to c), the drawing of the head strap fully extends the support means. Limbs of double hinged portion 172 are overlaid such that the constricted portion 176 of strut element 175 engages the recess in the aperture of its first limb 174 and latches the respirator in its ready position.

Referring now to FIGS. 2 a) and FIGS. 5 c) to a), outward folding of the filter canisters fully collapses the support elements. First limb of portions 171 and 172 lie substantially parallel to each other and their second limbs. Strut element 175 is retained in aperture of the first limb 174 of double hinged portion 172 by the tab portion 177.

Referring now to FIG. 2 a) and FIGS. 6 a) and b) it will be apparent that outward folding of the filter canisters 16 provide a space there between for retention of a collapsed oro-nasal mask 6, collapsed dead space 151, hood 18 and head straps 20 by packaging means 21.

The compact, packaged respirator 13 is about the size of a video cassette and is conveniently carried or stored, for example, in a desk drawer. In the event of an emergency, the respirator is conveniently at hand and the packaging is removed. The collapsed oro-nasal mask 6 and dead space 152 recover their original form partially extending split hinged bracket 17. The hood 18 is drawn over the end and the oro-nasal mask 6 placed over the nose and mouth. Tightening of the head strap 20 over the hood draws the filter canisters 16 towards the ears and fully extends split hinged bracket 17 so as to provide sealing forces to the oro-nasal mask as described above. The wearer can safely exit the affected area.

1. An oro-nasal mask for use in a respirator, which mask comprises a resilient material and is capable of recovering its original shape on release from a collapsed form.

2. A mask according to claim 1, further comprising one or more rigid support elements at or adjacent the peripheral edge of the mask which are effective to transmit sealing forces to substantially the whole of the peripheral edge of the mask in use.

3. A mask according to claim 2, in which a rigid support element is provided on opposing lateral sides of the mask.

4. A mask according to claim 1, including integrally formed air inlet guidance and air outlet guidance means.

5. A mask according to claim 1, in which the resilient material comprises silicone rubber.

6. A respirator including the oro-nasal mask of claim 1.

7. A respirator including the oro-nasal mask of claim 3, in which the support element comprises a filter canister.

8. A respirator according to claim 7, in which the support element further comprises means permitting folding of the filter canisters.

9. A respirator according to claim 8, in which the folding means permit folding such that the major axis of each filter canister lies substantially within the same plane.

10. A respirator according to claim 6, in which the air outlet guidance means includes a one-way valve and defines a collapsible dead space.

11. A respirator according to claim 6, further comprising a collapsible hood.

12. A respirator according to claim 11, in which the hood comprises a transparent, chemical agent resistant material.

13. A package comprising the respirator of claim 6 in which the respirator is held in compact form.

14. (canceled)
